(19) United States
(12) Patent Application Publication
(10) Pub. No.: US 2006/0264624 A1
(43) Pub. Date: Nov. 23, 2006

(54) Methods for synthesizing imidazotriazinones

(76) Inventors: Alexander Heim-Riether, New York, NY (US); David Paul Rotella, Newtown, PA (US)

Correspondence Address: LEXICON GENETICS INCORPORATED, 8800 TECHNOLOGY FOREST PLACE, THE WOODLANDS, TX, 77381-1160, US

(21) Appl. No.: 11/436,111

(22) Filed: May 17, 2006

Related U.S. Application Data
(60) Provisional application No. 60/683,135, filed on May 20, 2005.
(60) Provisional application No. 60/699,777, filed on Jul. 15, 2005.

(51) Int. Cl. C07D 487/04

(52) U.S. Cl. 544/183

(57) ABSTRACT

Methods of synthesizing imidazotriazinones, such as vardenafil, and compositions useful for the same are disclosed.

This application claims priority to U.S. provisional application Nos. 60/683,135, filed May 20, 2005, and 60/699,777, filed Jul. 15, 2005, the entireties of which are incorporated herein by reference.

### 1. FIELD OF THE INVENTION

This invention relates to methods of synthesizing imidazo[5,1-f][1,2,4]triazinones, and compositions useful for the same.

### 2. BACKGROUND

Imidazo[5,1-f][1,2,4]triazinones, as isosteres of purine, are of interest for pharmaceutical research and human therapy. For example, the imidazo[5,1-f][1,2,4]triazinone scaffold 1 has recently received attention as the core structure of vardenafil 2, a potent and effective PDE5 inhibitor for the treatment of erectile dysfunction sold in the United States under the tradename LEVITRA. (Haning, H. et al., Bioorg. Med. Chem. Lett. 2002, 12, 865; U.S. Pat. No. 6,362,178; PCT WO02/50076; Dunn, P. J. Org. Process Res. Dev. 2005, 9, 88.)

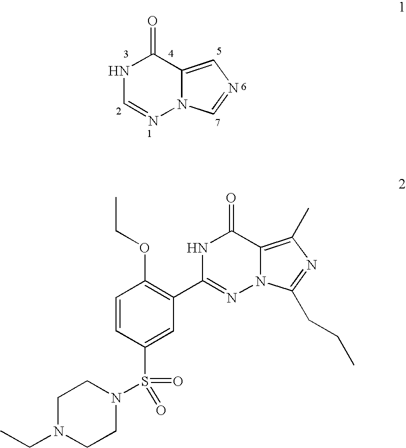

Analogs of this heterocycle containing nitrogen in the ring junction have also been described as muscle relaxants, bronchodilators, C-nucleoside isosteres, and purine analogs. (See, e.g., Chem. Abstr. 1973, 79, 53376; Chem. Abstr. 1978, 89, 15442; Chem. Abstr. 1974, 81, 120704; Marshall, D. Chem. Ind. 1983, 331; Mitchell, W. L. et al., J. Heterocycl. Chem. 1984, 21, 697; Knutsen, L. J. S. et al., J. Chem. Soc., Perkin Trans. 1 1985, 621; Knutsen, L. J. S. et al., J. Chem. Soc., Perkin Trans. 1 1984, 229; Bhattacharya, B. et al., J. Heterocycl. Chem. 1993, 30, 1341; Clarke, R. W. et al., J. Chem. Soc., Perkin Trans. 1 1979, 1120.)

Reported methods of preparing imidazo[5,1-f][1,2,4]triazinones typically require multiple synthetic steps, which can be time consuming and costly. For example, they can reduce the yield of the final product and produce waste products requiring specialized handling and disposal.

A conventional method of preparing these compounds is shown below: 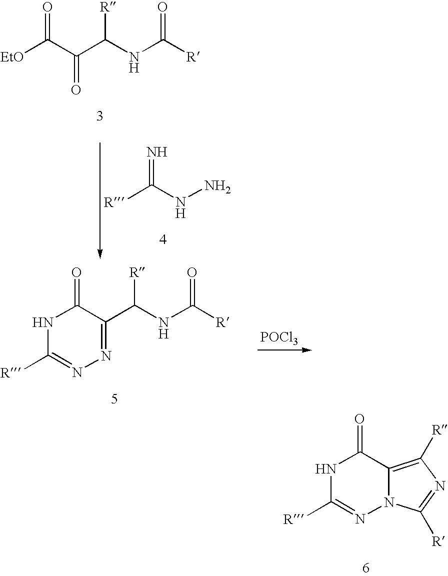
(Charles, I. ept al., J. Chem. Soc., Perkin Trans. 1 1980, 1139.) This approach begins with a Dakin-West reaction of acylated α-aminoacids and ethyl oxalyl chloride to afford acylamino-α-ketoester 3. Ketoester 3 is typically used without isolation to react with amidrazones 4, which are prepared in situ from the corresponding amidine and hydrazine. This condensation results in the triazinone-core 5 in moderately low yields of 13-26 percent over two steps. Triazinone 5 can then be cyclized to the imidazo[5,1-f][1,2,4]triazinone 6 in the presence of phosphoryl chloride.

The synthetic approach shown above was reportedly used both for the medicinal chemistry and chemical development of vardenafil (2). (Haning, H. et al., Bioorg. Med. Chem. Lett. 2002, 12, 865; U.S. Pat. No. 6,362,178; PCT WO02/50076; Dunn, P. J. Org. Process Res. Dev. 2005, 9, 88.) In both cases, reactive intermediates such as ketoester 3 (R₁=n-propyl, R₂=Me) complicate the synthesis. The overall yield of the vardenafil intermediate 9, shown below, over the five steps was 30 and 50 percent, respectively.

An alternative route to the vardenafil intermediate 9 has also been reported: 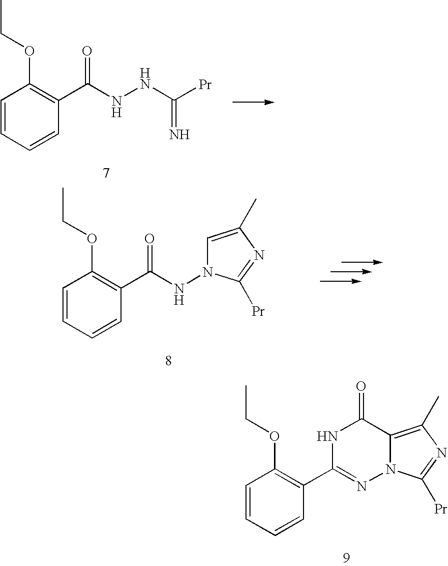
(PCT WO02/50075.) This approach avoids the most reactive intermediate 3 by forming the imidazole 8, followed by the cyclization to imidazotriazinone 9. Still, this approach only provides a reported yield of 19 percent over five steps. Thus, a need exists for improved methods of preparing imidazo[5,1-f][1,2,4]triazinones.

### 3. SUMMARY OF THE INVENTION

This invention encompasses novel methods of preparing imidazotriazinone compounds, as well as compositions that can be used in their synthesis.

One embodiment of the invention encompasses a method of preparing an imidazotriazinone, which comprises contacting a compound of Formula II: 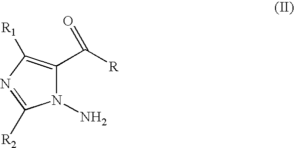
wherein R is alkoxy or hydroxy; R₁ is hydrogen, halogen, alkoxy, nitro, nitrile, optionally substituted alkyl, alkenyl, alkynyl, aryl, aralkyl, or heterocycle; and R₂ is hydrogen, halogen, alkoxy, nitro, nitrile, optionally substituted alkyl, alkenyl, alkynyl, aryl, aralkyl, or heterocycle; with a compound of formula IV or V 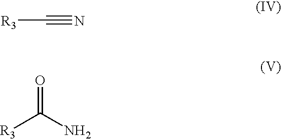
wherein R₃ is hydrogen, optionally substituted lower alkyl, or —N(R₄)(R₅), wherein R₄ and R₅ are individually hydrogen or optionally substituted lower alkyl; under conditions sufficient for the cyclization of the compound of Formula II.

Another embodiment of the invention encompasses a method of preparing an imidazotriazinone, which comprises contacting a compound of Formula II: 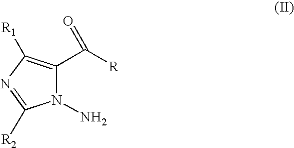
wherein R is amino; R₁ is hydrogen, halogen, alkoxy, nitro, nitrile, optionally substituted alkyl, alkenyl, alkynyl, aryl, aralkyl, or heterocycle; and R₂ is hydrogen, halogen, alkoxy, nitro, nitrile, optionally substituted alkyl, alkenyl, alkynyl, aryl, aralkyl, or heterocycle; with a compound of formula VI: 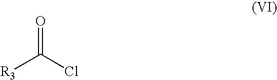
wherein R₃ is hydrogen, optionally substituted alkyl, alkoxy, alkenyl, alkynyl, aryl, aralkyl or heteroaryl, under conditions sufficient for the cyclization of the compound of Formula II.

In particular embodiments of the invention, the compound of formula II is prepared by the N-amination of a compound of Formula I:

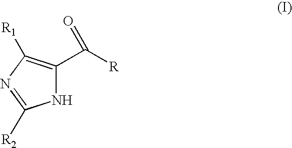

Another embodiment of the invention encompasses a method of preparing vardenafil (2), which comprises: contacting 3-(2-ethoxy-benzoylamino)-5-methyl-2-propyl-3H-imidazole-4-carboxamide with conditions sufficient for the formation of 2-(2-ethoxy-phenyl)-5-methyl-7-propyl-3H-imidazo[5,1-f][1,2,4]triazin-4-one; chlorosulphonation of 2-(2-ethoxy-phenyl)-5-methyl-7-propyl-3H-imidazo[5,1-f][1,2,4]triazin-4-one to obtain 4-ethoxy-3-(5-methyl-4-oxo-7-propyl-3,4-dihydro-imidazo[5,1-f][1,2,4]triazin-2-yl)-benzenesulfonic acid; and contacting 4-ethoxy-3-(5-methyl-4-oxo-7-propyl-3,4-dihydro-imidazo[5,1-f][1,2,4]triazin-2-yl)-benzenesulfonic acid with conditions suitable for the formation of vardenafil (2). A specific method further comprises contacting 3-amino-5-methyl-2-propyl-3H-imidazole-4-carboxamide with 2-ethoxy-benzoyl chloride under conditions sufficient for the formation of the 3-(2-ethoxy-benzoylamino)-5-methyl-2-propyl-3H-imidazole-4-carboxamide.

Another embodiment of the invention encompasses a method of synthesizing a 7-aryl-imidazotriazinone, which comprises the bromination of an unsubstituted imidazotriazinone followed by a Suzuki coupling.

This invention also encompasses compounds of formulas I, II and III, and compositions comprising them. Specific compounds encompassed by the invention are compounds of Formula II.

### 4. DETAILED DESCRIPTION

This invention encompasses novel synthetic methods of preparing imidazo[5,1-f][1,2,4]triazinone compounds such as, but not limited to, vardenafil.

One embodiment of the invention is represented below in Scheme 1: 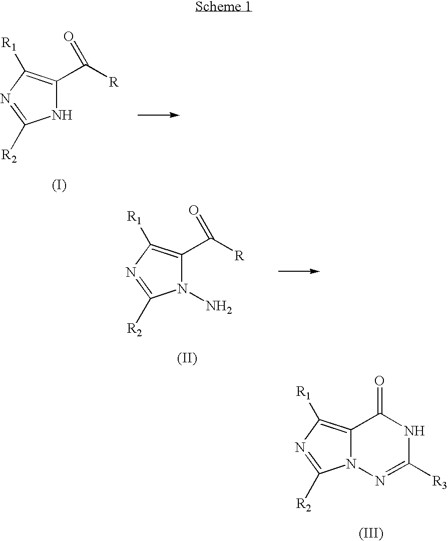
wherein R is alkoxy (e.g., —OMe, —OEt) or amine (e.g., —NH₂); R₁ is hydrogen, halogen, alkoxy, nitro, nitrile, optionally substituted alkyl, alkenyl, alkynyl, aryl, aralkyl, or heterocycle; and R₂ is hydrogen, halogen, alkoxy, nitro, nitrile, optionally substituted alkyl, alkenyl, alkynyl, aryl, aralkyl, or heterocycle; and R₃ is hydrogen, —N(R₄)(R₅), optionally substituted alkyl, alkenyl, alkynyl, hydroxy, alkoxy, aryl, aralkyl or heteroaryl, wherein R₄ and R₅ are individually hydrogen or optionally substituted alkyl, aryl, aralkyl or heteroaryl.

Unless otherwise indicated, the term “alkyl” means a saturated straight chain, branched or cyclic (“cycloalkyl”) hydrocarbon having from 1 to 20 (e.g., 1 to 10 or 1 to 4) carbon atoms. Alkyl moieties having from 1 to 4 carbons are referred to as “lower alkyl.” Examples of alkyl groups include, but are not limited to, methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl and dodecyl. Examples of cycloalkyl groups include, but are not limited to, cyclopropyl, cyclobutyl, cyclopentyl, cyclohexyl, and adamantyl. As used herein, the term “alkyl” includes “alkenyl” and “alkynyl” moieties.

Unless otherwise indicated, the term “alkenyl” means a straight chain, branched or cyclic hydrocarbon having from 2 to 20 (e.g., 2 to 10 or 2 to 6) carbon atoms, and including at least one carbon-carbon double bond. Representative alkenyls moieties include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl and 3-decenyl.

Unless otherwise indicated, the term “alkynyl” means a straight chain, branched or cyclic hydrocarbon having from 2 to 20 (e.g., 2 to 20 or 2 to 6) carbon atoms, and including at least one carbon-carbon triple bond. Representative alkynyl moieties include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl and 9-decynyl.

Unless otherwise indicated, the term “alkoxy” means an —O-alkyl group. Examples of alkoxy groups include, but are not limited to, —OCH₃, —OCH₂CH₃, —O(CH₂)₂CH₃, —O(CH₂)₃CH₃, —O(CH₂)₄CH₃, and —O(CH₂)₅CH₃.

Unless otherwise indicated, the term “aryl” means an aromatic monocyclic or polycyclic ring or ring system composed of carbon and hydrogen atoms. An aryl moiety may comprise two or more rings bound to one another by single bonds or fused together. Examples include, but are not limited to, phenyl, biphenyl, tolyl, anthracenyl, fluorenyl, indenyl, azulenyl, phenanthrenyl and naphthyl. A specific aryl moiety is phenyl.

Unless otherwise indicated, the term “arylalkyl” means an aryl moiety bound to an alkyl moiety.

Unless otherwise indicated, the term “halogen” or “halo” refers to fluorine, chlorine, bromine, or iodine.

Unless otherwise indicated, the term “heteroalkyl” refers to an alkyl moiety in which one or more carbon atoms is replaced by a heteroatom (e.g., N, O or S).

Unless otherwise indicated, the term “heteroaryl” means an aryl moiety where at least one of its carbon atoms has been replaced with a heteroatom (e.g., N, O or S). Examples include, but are not limited to, acridinyl, benzimidazolyl, benzofuranyl, benzoisothiazolyl, benzoisoxazolyl, benzoquinazolinyl, benzothiazolyl, benzoxazolyl, furyl, imidazolyl, indolyl, isothiazolyl, isoxazolyl, oxadiazolyl, oxazolyl, phthalazinyl, pyrazinyl, pyrazolyl, pyridazinyl, pyridyl, pyrimidinyl, pyrimidyl, pyrrolyl, quinazolinyl, quinolinyl, tetrazolyl, thiazolyl and triazinyl.

Unless otherwise indicated, the term “heteroarylalkyl” means a heteroaryl moiety bound to an alkyl moeity.

Unless otherwise indicated, the term “heterocycle” refers to an aromatic, partially aromatic or non-aromatic, monocyclic or polycyclic ring or ring system comprised of carbon, hydrogen and at least one heteroatom (e.g., N, O or S). A heterocycle may comprise two or more rings bound to one another by single bonds or fused together. Heterocycles include heteroaryls. Examples include, but are not limited to benzo[1,3]dioxolyl, 2,3-dihydro-benzo[1,4]dioxinyl, cinnolinyl, furanyl, hydantoinyl, morpholinyl, oxetanyl, oxiranyl, piperazinyl, piperidinyl, pyrrolidinonyl, pyrrolidinyl, tetrahydrofuranyl, tetrahydropyranyl, tetrahydropyridinyl, tetrahydropyrimidinyl, tetrahydrothiophenyl, tetrahydrothiopyranyl and valerolactamyl.

Unless otherwise indicated, the term “substituted,” when used to describe a chemical structure or moiety, refers to a derivative of that structure or moiety wherein one or more of its hydrogen atoms is substituted with a chemical moiety or functional group such as, but not limited to, alcohol (e.g., hydroxyl, alkyl-OH), aldehylde, alkanoyloxy, alkoxycarbonyl, alkyl (e.g., methyl, ethyl, propyl, t-butyl), alkenyl, alkynyl, amide, amine (primary, secondary and tertiary such as alkylamino, arylamino, arylalkylamino), aroyl, aryl, aryloxy, azo, carbamyl (e.g., CONH₂, as well as CONH-alkyl, CONH-aryl, and CONH-arylalkyl), carbonyl, carboxyl, carboxylic acid, carboxylic acid anhydride, carboxylic acid chloride, cyano, ester, epoxide, ether (e.g., methoxy, ethoxy), guanidino, imine (primary and secondary), isocyanate, isothiocyanate, ketone, halo, haloalkyl (e.g., fluoromethyl, difluoromethyl, trifluoromethyl), hemiacetal, heterocycle, nitrile, nitro, phosphodiester, sulfide, sulfonamido (e.g., SO₂NH₂), sulfone, sulfonyl (including alkylsulfonyl, arylsulfonyl and arylalkylsulfonyl), sulfoxide and thiol (e.g., sulfhydryl, thioether).

Unless otherwise indicated, the term “include” has the same meaning as “include, but are not limited to.” Similarly, the term “includes” has the same meaning as “includes, but is not limited to.”

Unless otherwise indicated, an adjective before a series of nouns is to be construed as applying to each of the nouns. For example, the phrase “optionally substituted alky, aryl, or heteroaryl” has the same meaning as “optionally substituted alky, optionally substituted aryl, or optionally substituted heteroaryl.”

It should be noted that if there is a discrepancy between a depicted structure and a name given that structure, the structure should be accorded more weight. In addition, if the stereochemistry of a structure or a portion of a structure is not indicated with, for example, bold or dashed lines, the structure or the portion of the structure is to be interpreted as encompassing all stereoisomers of it.

Referring to Scheme 1, above, the N-amination of compound I to afford compound II can be achieved using reagents known in the art, such as O-acylhydroxylamines and O-sulfonylhydroxylamines. Examples include hydroxylamine-O-sulfonic acid, O-(2,4-dinitrophenyl)-hydroxylamine, O-(diphenylphosphinyl)hydroxylamine, O-mesitylenesulfonylamine, O-p-toluenesulfonylamine and O-mesitoylhydroxylamine. In a preferred embodiment, compound I is contacted with a suitable base (e.g., butyl lithium, LDA (lithium diisopropylamide), LHMDS (lithium hexamethyldisilazide), KHMDS (potassium hexamethyldisilazide)) at a suitable temperature (e.g., −78-25° C., preferable 0-25° C.), followed by treatment with O-(diphenylphosphinyl)hydroxylamine. A preferred base is LHMDS (lithium hexamethyldisilazide) in a suitable solvent, such as NMP (N-methyl pyrrolidinone) or DMF (dimethyl formamide). (See, e.g., Belley, M. et al., Synlett 2001, 222.) The selection of reagents and reaction conditions are preferably adjusted to minimize the formation of IIa:

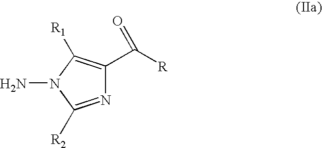

Compounds of formula II are readily cyclized with formamide to afford compounds of formula III wherein R₃ is hydrogen. For example, each of the three substituted imidazoles (10), (13) and (16) shown below were N-aminated to provide the corresponding aminoimidazoles (11), (14) and (17), which were then cyclized to obtain the corresponding imidazotriainones (12), (15) and (18):

| Imidazole | N-Aminoimidazole | Yield | Imidazotriazinone | Yield |
|---|---|---|---|---|
| 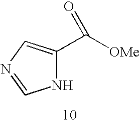 | 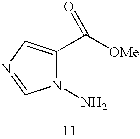 | 64% | 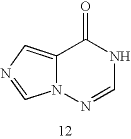 | 52% |
| 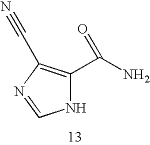 | 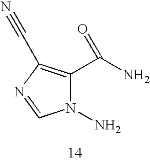 | 65% | 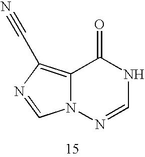 | 83% |
| 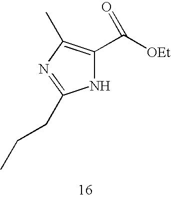 | 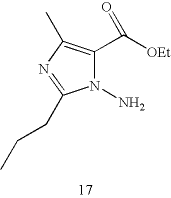 | 75% | 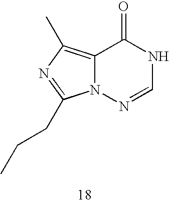 | 68% |

In these reactions, the use of DMF instead of NMP as the solvent did not influence yield, but did facilitate product isolation. NMP can require extensive extraction with an organic solvent (EtOAc, Et₂O, CH₂Cl₂) after the reaction is quenched with water. Running the reaction in DMF allows evaporation of the solvent in vacuo. The remaining solid can be efficiently extracted with dichloromethane to give the product in greater than 90% purity. In case of compounds with poor solubility in organic solvents such as N-aminoimidazole (14), filtration of the reaction mixture to remove the formed LiOP(Ph)₂O was carried out without quenching the reaction with water. Upon concentration of the filtrate in vacuo, the product precipitates and can be obtained by filtration in greater than 95 percent purity. The cyclization of the N-aminoimidazoles shown above with formamide proceeded in good yield.

Compounds of formula III wherein R₃ is something other than hydrogen can also be prepared. This invention encompasses various methods of their preparation. In one, substituents are incorporated using synthetic routes previously applied to the synthesis of bicyclic derivatives of pyrimidinone (e.g. furo-, thieno-, pyrrolo[2,3-d]pyrimidines). One route utilizes the reaction of enamino esters with nitriles in the presence of acid. (See, e.g., Dave, K. G. et al., J. Het. Chem. 1980, 17, 1497). Without being limited by theory, this proceeds via the formation of an amidine intermediate, which undergoes intramolecular cyclization by nucleophilic attack on the carbonyl function under the acidic reaction conditions or under subsequent treatment with base. (Venugopalan, B. et al., J. Het. Chem. 1988, 25, 1633).

This approach can be used to afford compounds of formula III, as shown below: 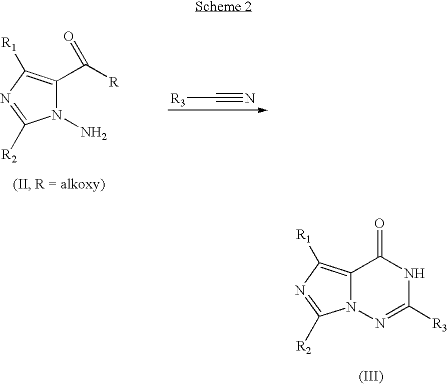
But while this approach works well if R₃ is relatively small (e.g., methyl, NH₂), the preparation of sterically hindered triazinones (e.g., compounds wherein R₃ is a bulky moiety, such as phenyl or 2-ethoxybenzonitrile) is accomplished using an alternative route.

In one approach, the compound of formula II is acylated (e.g., with benzoyl chloride), and then contacted with ammonium hydroxide under pressure and at a temperature sufficient to afford the desired product. An example of this approach is shown below:

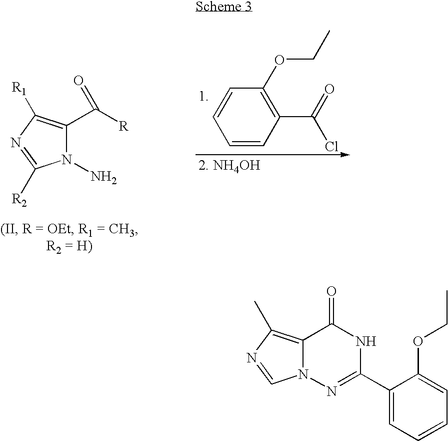

In a more preferred route, the R moiety, not ammonium hydroxide, provides the source of nitrogen. This approach may be used for the preparation of triazinones optionally substituted with a variety of small and large (sterically bulky) moieties. Its use in the synthesis of vardenafil is shown below:

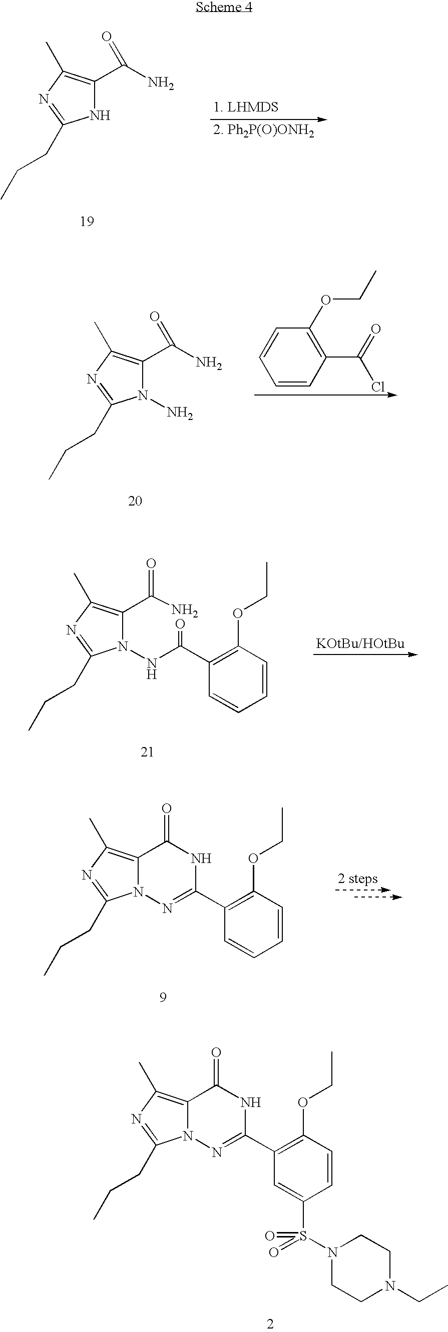

In carrying out the synthesis shown above, compound 19 was N-aminated to obtain compound 20 in a 41 percent yield. Acylation of compound 20 with 2-ethoxybenzoyl chloride provided the 3-(2-ethoxy-benzoylamino)-imidazole 21 in a 52 percent yield. Compound 21 was then contacted with a base (ten equivalents of potassium tert-butoxide in tert-butanol) in a sealed tube at 160° C. (See Dale, D. J. et al., Org. Process Res. Dev. 2000, 4, 17). Cyclization to the imidazotriazinone 9 proceeded in a 72 percent yield and successfully completed the formal synthesis of vardenafil 2 in three steps. Although this particular example of the invention provided vardenafil in an overall yield of 15 percent, it is expected that higher yields may be obtained upon optimization of the procedure.

However compounds of formula III are prepared, they may be further functionalized. An example of this is shown below:

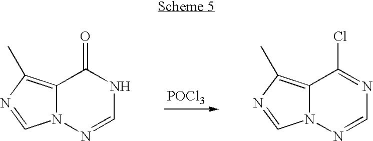

Imidazotriazinones substituted at the 7-position may also be prepared using methods of the invention. In this regard, the invention encompasses methods of preparing compounds of formula III substituted at the 2-position.

In one embodiment, a compound of formula III (R₂ is hydrogen) is brominated to afford a compound of formula VII: 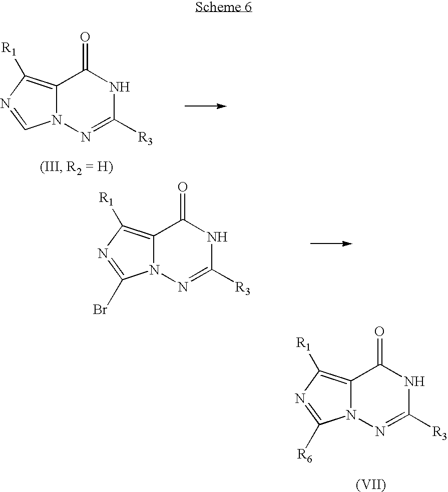
where R₆ is aryl, heteroaryl, alkenyl, alkynyl, carboxyl, or carboxamide. The brominated intermediate is a useful synthon for C—C-coupling conditions. For example, standard Suzuki coupling (see, e.g., Gong, Y.; He, W. Org. Lett. 2002, 4, 3803) provided 7-phenyl-imidazotriazinone in a 59 percent yield, as shown below:

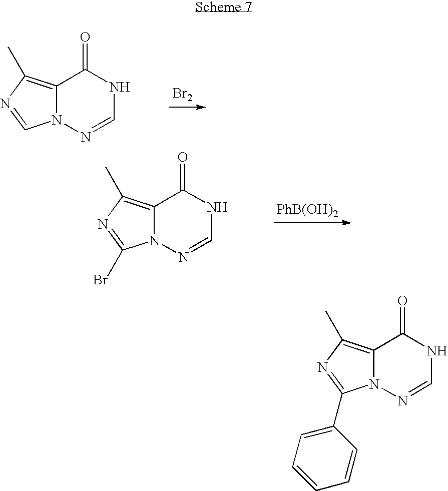

### 5. EXAMPLES

Aspects of this invention may be understood from the following examples, which do not limit its scope.

All reactions involving water-sensitive chemicals were carried out in oven-dried glassware with magnetic stirring under nitrogen. Anhydrous solvents were purchased from Aldrich (St Louis, Mo.). Other chemicals were either commercially available or prepared according to the cited references. Microwave reactions were carried out in an Emrys Optimizer XP instrument. ¹H and ¹³C NMR spectra were recorded with a Bruker ARX 300 or Varian Mercury 400 in various deuterated solvents at 303° K, and chemical shifts are reported relative to the distinguished solvent signals. Combustion analyzes were conducted by Robertson Microlit (Madison, N.J.). EI mass spectrometry was performed on Waters or Shimadzu LC/MS instruments. TLC was performed on glass silica plates (0.2 mm silica gel 60 F₂₅₄). Detection was done by UV or coloration with ninhydrin. Flash chromatography was performed on silica gel 60 (230-400 mesh) on an ISCO SQ-16× with the eluent given in brackets. All solvents used were HPLC grade.

### 5.1. Example 1

### Ethyl 5-Methyl-2-propyl-3H-imidazole-4-carboxylate

To a stirred suspension of ethyl butaneimidate hydrochloride (24.0 g, 0.16 mol) and triethylamine (32 mL, 0.23 mol) in absolute EtOH (200 mL) a solution of ethyl 2-amino-3-oxobutanoate hydrochloride (11.6 g, 0.06 mol) in absolute EtOH (100 mL) was added dropwise over 1 h. After stirring overnight under an atmosphere of N₂, the orange reaction mixture was concentrated in vacuo to ˜50 ml. The precipitating TEA hydrochloride was filtered of and the remaining solution concentrated in vacuo to produce an orange oil. Purification by chromatography eluting with 40% increasing to 75% ethyl acetate in hexane gave the title compound as a white solid (7.65 g, 65%). ¹H NMR (75 MHz, CDCl₃):δ 4.27 (q, J=6.8 Hz, 2H), 2.65 (t, J=6.9 Hz, 2H), 2.46 (s, 3H), 1.69 (sextet, J=6.9 Hz, 2H), 1.24 (t, J=6.9 Hz, 3H), 0.89 (t, J=6.9 Hz, 3H). (See European Patent EP0514216A1, 1992; Chem. Abstr. 1993, 118, 169107, and see also Judd, D. B., et al., J. Med. Chem. 1994, 37, 3108).

### 5.2. Example 2

### Production of 5-methyl-2-propyl-3H-imidazole-4-carboxamide

Ethyl 5-Methyl-2-propyl-3H-imidazole-4-carboxylate (1.49 g, 7.59 mmol) in concentrated ammonium hydroxide (20 mL) was stirred at 130° C. for 24 h in a sealed tube. The solvent was removed in vacuo and the remaining solid was purified by flash chromatography on silica gel eluting with 2% increasing to 10% methanol in dichloromethane. The product was obtained as a white solid (671 mg, 53%). R_{f}=0.44 (10% MeOH in DCM), ¹H NMR (75 MHz, [D₆]-DMSO): δ 11.86 (s_{br}, 1H), 6.99 (s_{br}, 1H), 6.80 (s_{br}, 1H), 2.50 (t, J=7.5 Hz, 2H), 2.38 (s, 3H), 1.63 (sextet, J=7.4 Hz, 2H), 0.88 (t, J=7.3 Hz, 3H). ¹³C NMR (75 MHz, [D₆]-DMSO): δ 166.1, 145.6, 130.5, 129.6, 30.1, 21.6, 13.9, 10.9. MS, m/z (%) 168.0 (100) [M⁺+1]. Anal. Calcd for C₈H₁₃N₃O (167.21): C 57.47, H 7.84, N 25.13. Found: C, 57.80; H, 8.59; N, 24.96.

### 5.3. Example 3

### General procedure for N-amination of imidazoles

Lithium hexamethyldisilazane (1.10 mL of a 1M solution in THF, 1.1 mmol) was slowly added to the imidazole (1.0 mmol) in anhydrous DMF (10 mL) at −10° C. After stirring for 10 min, O-diphenylphosphinyl)hydroxylamine (280 mg, 1.2 mmol) was added at 0° C., followed by stirring at room temperature for 4 h-6 h (in cases where the reaction mixture becomes too viscous additional DMF was added). The reaction was quenched with water until a clear solution was formed and concentrated to dryness under reduced pressure. The residue was washed several times with ethyl acetate or dichloromethane. The combined organic fractions were concentrated in vacuo and purified by flash chromatography on silica gel.

### 5.4. Example 4

### Production of Ethyl 3-Amino-5-methyl-3H-imidazole-4-carboxylate

N-Amination of ethyl 5-methyl-3H-imidazole-4-carboxylic acid (5.39 g, 34.96 mmol) following the general procedure. Purification by flash chromatography on silica gel eluting with 0% increasing to 4% methanol in dichloromethane gave a yellow-white solid (4.23 g, 71%). R_{f} 0.53 (7% MeOH in DCM), ¹H NMR (300 MHz, CDCl₃): δ 7.48 (s, 1H), 5.33 (s, 2H), 4.27 (q, J=6.9 Hz, 2H), 2.35 (s, 3H), 1.31 (t, J=6.9 Hz, 3H). ¹³C NMR (75 MHz, CDCl₃): δ 61.9 (CO₂Et), 146.6, 140.4 (CH), 118.4, 60.8, 16.4, 14.7. MS, m/z (%) 170 (100) [M⁺+1] Anal. Calcd for C₇H₁₁N₃O₂ (169.18): C, 49.70; H, 6.55; N, 24.84. Found: C 49.85, H 6.45, N 24.74.

### 5.5. Example 5

### Production of Ethyl 1-Amino-5-methyl-1H-imidazole-4-carboxylate

N-Amination of ethyl 5-methyl-3H-imidazole-5-carboxylate (5.39 g, 34.96 mmol) following the general procedure. Purification by flash chromatography on silica gel eluting with 0% increasing to 4% methanol in dichloromethane gave a yellow-waxy solid (1.29 g, 22%). R_{f} 0.18 (7% MeOH in DCM), ¹H NMR (300 MHz, CDCl₃): δ 7.38 (s, 1H), 4.79 (s_{br}, 2H), 4.25 (q, J=7.2 Hz, 2H), 2.44 (s, 3H), 1.30 (t, J=7.2 Hz, 3H). ¹³C NMR (75 MHz, CDCl₃): δ 164.1 (CO₂Et), 137.6 (CH), 137.2, 127.3, 60.6, 14.8, 9.5.

### 5.6. Example 6

### Production of Methyl 3-Amino-3H-imidazole-4-carboxylate

N-Amination of methyl 3H-imidazole-4-carboxylate (1.0 g, 7.93 mmol) following the general procedure. Purification by flash chromatography on silica gel eluting with 50% increasing to 100% ethyl acetate in hexane gave a white solid (0.72 g, 64%). ¹H NMR (300 MHz, CDCl₃): δ 7.65 (s, 1H), 7.59 (s, 1H), 5.42 (s, 2H), 3.84 (s, 3H). ¹³C NMR (75 MHz, CDCl₃): δ 161.5 (CO₂Et), 142.1 (CH), 135.9 (CH), 122.0, 52.0. MS, m/z (%) 142 (100) [M⁺+1] Anal. Calcd for C₅H₇N₃O₂ (141.13): C, 42.55; H, 5.00; N, 29.77. Found: C 42.59, H 4.89, N 29.88.

### 5.7. Example 7

### 3-Amino-5-cyano-3H-imidazole-4-carboxamide

N-Amination of 5-cyano-3H-imidazole-4-carboxamide (200 mg, 1.469 mmol) following the general procedure. After 6 h the reaction mixture was filtered to remove the formed lithium salt of O-diphenylphosphinic acid without quenching the reaction. Evaporation of the solvent in vacuo led to precipitation of product. The product was collected by filtration and washed with MeOH to give a pale yellow solid (145 mg, 65%).

¹H NMR (300 MHz, [D₆]-DMSO): δ 8.35 (s_{br}, 1H), 8.07 (s_{br}, 1H), 7.92 (s, 1H), 6.72 (s, 2H). ¹³C NMR (75 MHz, [D₆]-DMSO): δ 185.3 (CONH₂), 142.1 (CH), 133.4, 115.2, 113.9. MS, m/z (%) 152 (100) [M⁺+1] Anal. Calcd for C₅H₅N₅O (151.13): C, 39.74; H, 3.33; N, 46.34. Found: C, 39.82; H, 3.12; N, 46.56.

### 5.8. Example 8

### Production of Ethyl 3-Amino-5-methyl-2-propyl-3H-imidazole-4-carboxylate

N-Amination of ethyl 5-methyl-2-propyl-3H-imidazole-4-carboxylate (2.0 g, 10.19 mmol) following the general procedure. Purification by flash chromatography on silica gel eluting with 50% increasing to 75% ethyl acetate in hexane gave a yellow-white solid (1.63 g, 75%). ¹H NMR (300 MHz, CDCl₃): δ 4.95 (s, 2H), 4.01 (q, J=7.0 Hz, 2H), 2.43 (t, J=7.5 Hz, 3H), 2.10 (s, 3H), 1.42 (sextet, J=7.5 Hz, 2H), 1.07 (t, J=7.0 Hz, 3H), 0.66 (t, J=7.5 Hz, 3H). ¹³C NMR (75 MHz, CDCl₃): δ 162.2, 152.4, 145.2, 117.6, 60.4, 28.3, 21.5, 16.2, 14.7, 14.2. MS, m/z (%) 212 (100) [M⁺+1] Anal. Calcd for C₁₀H₁₇N₃O₂ (211.25): C 56.85, H 8.11, N 19.89. Found: C, 56.62; H, 7.85; N, 19.97.

### 5.9. Example 9

### Production of 3-Amino-5-methyl-2-propyl-3H-imidazole-4-carboxamide

N-Amination of 3-amino-5-methyl-2-propyl-3H-imidazole-4-carboxylamide (583 mg, 3.48 mmol) following the general procedure. Purification by flash chromatography on silica gel eluting with 4% increasing to 10% methanol in dichloromethane gave a pale yellow waxy solid (259 mg, 41%). R_{f} 0.32 (10% MeOH in DCM), ¹H NMR (300 MHz, CD₃OD): δ 2.72 (t, J=7.5 Hz, 2H), 2.41 (s, 3H), 1.76 (sextet, J=7.5 Hz, 2H), 1.01 (t, J=7.5 Hz, 3H). ¹³C NMR (75 MHz, CD₃OD): δ 165.1, 151.9, 141.9, 121.5, 28.4, 21.8, 15.1, 14.2. MS, m/z (%) 183.2 (100) [M⁺+1]. Anal. Calcd for C₈H₁₄N₄O (182.23): C, 52.73; H, 7.53; N, 30.75. Found: C 52.72, H 7.53, N 31.00.

### 5.10. Example 10

### Production of 1-Amino-5-methyl-2-propyl-1H-imidazole-4-carboxamide

N-Amination of 3-amino-5-methyl-2-propyl-3H-imidazole-4-carboxamide (583 mg, 3.48 mmol) following the general procedure. Purification by flash chromatography on silica gel eluting with 4% increasing to 10% methanol in dichloromethane gave a white solid (350 mg, 55%). R_{f} 0.44 (10% MeOH in DCM), ¹H NMR (300 MHz, CD₃OD): δ 2.75 (t, J=7.3 Hz, 2H), 2.50 (s, 3H), 1.75 (sextet, J=7.3 Hz, 2H), 1.00 (t, J=7.3 Hz, 3H). ¹³C NMR (CD₃OD): δ 168.5, 149.8, 136.4, 126.9, 28.9, 22.3, 14.1, 9.4.

### 5.11. Example 11

### General procedure for the preparation of Imidazo[5,1-f][1,2,4]triazin-4(3H)-ones

In a sealed tube, amino-imidazole (1.00 mmol) in formamide (1-2 mL) was heated at 180° C. for 2-8 h. In cooling down to RT, most imidazo[5,1-f][1,2,4]triazin-4(3H)-ones were precipitating out and could be isolated by filtration. The precipitate was washed with ethyl acetate. The same solvent was used to precipitate out the products if necessary. The products were obtained as white/beige solids (52-89% yield).

### 5.12. Example 12

### Production of 5-Methyl-3H-imidazo[5,1-f][[1,2,4]triazin-4-one

According to general procedure, ethyl 3-amino-5-methyl-3H-imidazole-4-carboxylate (2.50 g, 14.77 mmol) gave beige crystals (1.96 g, 89%). ¹H NMR (300 MHz, [D₆]-DMSO): δ 11.32 (s_{br}, 1H), 8.28 (s, 1H), 7.79 (s, 1H), 2.45 (s, 3H). ¹³C NMR (75 MHz, [D₆]-DMSO): δ 155.2, 140.8 (CH), 139.0, 133.1 (CH), 115.7, 14.7. MS, m/z (%) 150.9 (100) [M⁺+1] Anal. Calcd for C₆H₆N₄O (150.14): C, 48.00; H, 4.03; N, 37.32. Found: C, 48.25; H, 4.03; N, 37.29.

### 5.13. Example 13

### Production of 3H-imidazo[5,1-f][1,2,4]triazin-4-one

According to general procedure, methyl 3-amino-3H-imidazole-4-carboxylate (110 mg, 0.779 mmol) gave white crystals. (55 mg, 52%). ¹H NMR (300 MHz, [D₆]-DMSO): δ 11.87 (s_{br}, 1H), 8.47 (s, 1H), 7.93 (s, 1H), 7.78 (s, 1H). ¹³C NMR (75 MHz, [D₆]-DMSO): δ 154.2, 140.9 (CH), 135.0, 128.0 (CH), 120.4. MS, m/z (%) 137.0 (100) [M⁺+1] Anal. Calcd for C₅H₄N₄O (136.11): C, 44.12; H, 2.96; N, 41.16. Found: C, 43.91; H, 2.70; N, 41.26.

### 5.14. Example 14

### Production of 5-Cyano-3H-imidazo[5,1-f][1,2,4]triazin-4-one

According to general procedure, 3-amino-5-cyano-3H-imidazole-4-carboxyamide (96 mg, 0.653 mmol) gave the product (90% pure), which was purified by reversed phase HPLC, eluted with 0.1% TFA H₂O/MeOH gradient, to give the product as a beige-white solid. (87 mg, 83%). ¹H NMR (300 MHz, [D₆]-DMSO): δ 12.56 (s_{br}, 1H), 8.72 (s, 1H), 8.14 (s, 1H). ¹³C NMR (75 MHz, [D₆]-DMSO): δ 152.7, 142.8 (CH), 136.4 (CH), 126.9, 114.2, 109.2. MS, m/z (%) 183.90 (100) [M⁺+1+Na] Anal. Calcd for C₆H₃N₅O (161.12): C 44.73, H 1.88, N 43.47.

### 5.15. Example 15

### Production of 5-Methyl-2-propyl-3H-imidazo[5,1-f][1,2,4]triazin-4-one

According to general procedure, ethyl 3-amino-5-methyl-2-propyl-3H-imidazole-4-carboxylate (100 mg, 0.509 mmol) gave a white solid. (67 mg, 68%). ¹H NMR (300 MHz, [D₆]-DMSO): a 11.51 (s_{br}, 1H), 7.76 (s, 1H), 2.79 (t, J=7.2 Hz, 2H), 2.43 (s, 3H), 1.69 (sextet, J=7.2 Hz, 2H), 0.90 (t, J=6.0 Hz, 3H). ¹³C NMR (75 MHz, [D₆]-DMSO): δ 155.3, 144.4, 139.9, 137.9, 115.2, 27.4, 20.6, 14.6, 14.0. MS, m/z (%) 193.2 (100) [M⁺+1] Anal. Calcd for C₉H₁₂N₄O (192.22): C, 56.24; H, 6.29; N, 29.15. Found: C, 55.94; H, 6.42; N, 29.13.

### 5.16. Example 16

### 4-Chloro-5-methyl-imidazo[5,1-f][1,2,4]triazine

5-Methyl-3H-imidazo[5,1-f][1,2,4]triazin-4-one (500 mg, 3.33 mmol) was refluxed in phosphoryl chloride for 5 h. The reaction mixture was cooled to room temperature and poured on ice followed by extraction with dichloromethane (3×20 mL). The organic layer is washed with saturated Na₂CO₃-solution, brine and dried over MgSO₄. The solvent was removed in vacuo and the remaining solid was purified by flash chromatography on silica gel eluting with 75% ethyl acetate in hexane. 4-Chloro-5-methyl-imidazo[5,1-f][1,2,4]triazine was obtained as a beige-white solid (309 mg, 45%). R_{f} 0.51 (50% EtOAc in hexane), ¹H NMR (400 MHz, CDCl₃): δ 8.38 (s, 1H), 8.06 (s, 1H), 2.74 (s, 3H). ¹³C NMR (100 MHz, CDCl₃): δ 156.7, 146.9 (CH), 137.3, 131.3 (CH), 116.9, 15.6. MS, m/z (%) 168.9 (100) [M⁺+1] Anal. Calcd for C₆H₅ClN₄ (168.59): C 42.75, H 2.99, Cl 21.03, N 33.23. Found: C 42.98, H 3.09, Cl 20.87, N 33.09.

### 5.17. Example 17

### 2,5-Dimethyl-3H-imidazo[5,1-f][1,2,4]triazin-4-one

To a solution of ethyl 3-amino-5-methyl-3H-imidazole-4-carboxylate (200 mg, 1.18 mmol) in 10 mL acetonitrile was passed dry hydrogen chloride gas at room temperature for 30 min. The reaction mixture was stirred for 18 h. Upon concentration a white solid was crashing out and was filtered off. The solid was taken in absolute ethanol (20 ml) and 5% aqueous sodium hydroxide (5 mL) and refluxed for 6 h. The solvent was evaporated and the reaction mixture was dissolved in water and acidified with 6N HCl. The precipitate was filtrated and washed with water. After drying in vacuo the product was obtained as a white solid (172 mg, 89%). ¹H NMR (300 MHz, [D₆]-DMSO): δ 11.61 (s_{br}, 1H), 8.21 (s, 1H), 2.45 (s, 3H), 2.18 (s, 3H). ³C NMR (75 MHz, [D₆]-DMSO): δ 155.5, 149.5, 138.7, 132.6, 114.3, 18.4, 14.6. MS, m/z (%) 165.0 (100) [M⁺+1] Anal. Calcd for C₇H₈N₄O (164.17): C 51.21, H 4.91, N 34.13. Found: C, 51.21; H, 4.73; N, 34.08.

### 5.18. Example 18

### 2-Amino-5-methyl-3H-imidazo[5,1-f][1,2,4]triazin-4-one

To a solution of ethyl 3-Amino-5-methyl-3H-imidazole-4-carboxylic acid (145 mg, 0.857 mmol) and cyanamide (42 mg, 1.028 mmol) in 8 mL dioxane was added concentrated hydrochloric acid (0.5 mL). The reaction mixture was refluxed under nitrogen atmosphere for 24 h. The formed brown oil was separated from the reaction mixture and after the addition of a solution of sodium hydroxide (41 mg, 1.028 mmol) in 10 mL water it was heated at 100° C. for 3 h. The cooled mixture was acidified with hydrochloric acid (6N) and was purified by reversed phase HPLC, eluted with 0.1% TFA H₂O/MeOH gradient, to give the product as a white solid (40 mg, 28%). ¹H NMR (300 MHz, CD₃OD): δ 7.84 (s, 1H), 2.52 (s, 3H). ¹³C NMR (75 MHz, CD₃OD): δ 155.7, 153.9, 134.2, 127.9, 116.5, 11.1. MS, m/z (%) 166.1 (100) [M⁺+1] Anal. Calcd for C₆H₇N₅O (165.16): C, 43.64; H, 4.27; N, 42.40.

### 5.19. Example 19

### Production of 2-(2-Ethoxy-phenyl)-5-methyl-3H-imidazo[5,1-f][1,2,4]triazin-4-one

To a solution of ethyl 3-amino-5-methyl-3H-imidazole-4-carboxylate (100 mg, 0.591 mmol) in anhydrous pyridine (2 mL) 2-ethoxy-benzoyl chloride (130 mg, 0.709 mmol) was added. The reaction mixture was stirred under nitrogen for 2 h at 100° C. before it was transferred into a sealed tube. After addition of concentrated ammonium hydroxide (5 mL) the mixture was stirred at 110° C. for 24 h. To the cooled reaction mixture ethyl acetate was added and the organic layer was extracted with water, brine and dried over MgSO₄. After the solvent was removed in vacuo the crude product was purified by flash chromatography on silica gel eluting with 0% increasing to 5% methanol in dichloromethane. The product was obtained as a white solid (20 mg, 13%). ¹H NMR (300 MHz, CDCl₃): δ 9.97 (s_{br}, 1H), 8.08 (dd, ³J=8.0 Hz, ⁴J=1.8 Hz, 1H), 7.99 (s, 1H), 7.43 (t, J=7.8 Hz, 1H), 7.05 (t, J=7.8 Hz, 1H), 6.97 (d, J=8.0 Hz, 1H), 4.19 (q, J=6.9 Hz, 2H), 2.58 (s, 3H), 1.49 (t, J=6.9 Hz, 3H). ¹³C NMR (75 MHz, CDCl₃): δ 157.4, 154.9, 147.5, 141.4, 133.8, 133.5, 130.4, 122.2, 117.4, 113.5, 65.7, 15.1, 15.0. MS, m/z (%) 271.1 (100) [M⁺+1] Anal. Calcd for C₁₄H₁₄N₄O₂ (270.29): C, 62.21; H, 5.22; N, 20.73. Found: C, 62.03; H, 4.99; N, 20.47.

### 5.20. Example 20

### Production of 3-(2-Ethoxy-benzoylamino)-5-methyl-2-propyl-3H-imidazole-4-carboxamide

To a solution of 5-methyl-2-propyl-3H-imidazole-4-carboxamide (160 mg, 0.878 mmol) in anhydrous pyridine (10 mL) 2-ethoxy-benzoyl chloride (194 mg, 1.054 mmol) was added. The reaction mixture was stirred under nitrogen for 2 h at 60° C. before the solvent was removed in vacuo. Ethyl acetate was added followed by extraction with saturated sodium carbonate solution, brine and drying over MgSO₄. After the solvent was removed in vacuo the crude product was purified by flash chromatography on silica gel eluting with 2% increasing to 7% methanol in dichloromethane. The product was obtained as a white solid (150 mg, 52%). ¹H NMR (300 MHz, CDCl₃): a 10.64 (s_{br}, 1H), 8.10 (d, J=8.2 Hz, 1H), 7.45 (t, J=7.6 Hz, 1H), 7.02 (t, J=7.6 Hz, 1H), 6.97 (d, J=8.2 Hz, 1H), 5.63 (s_{br}, 2H), 4.24 (q, J=6.9 Hz, 2H), 2.53 (t, J=7.6 Hz, 2H), 2.36 (s, 3H), 1.68 (sextet, J=7.6 Hz, 2H), 1.52 (t, J=6.9 Hz, 3H), 0.89 (t, J=7.6 Hz, 3H). ¹³C NMR (75 MHz, CDCl₃): δ 166.3, 162.6, 157.9, 152.4, 139.9, 134.9, 133.1, 121.8, 121.0, 119.0, 113.0, 65.7, 28.4, 21.1, 15.7, 15.1, 14.3. MS, m/z (%) 331.1 (100) [M++l] Anal. Calcd for C₁₇H₂₂N₄O₃ (330.39): C, 61.80; H, 6.71; N, 16.96. Found: C, 61.39; H, 6.78; N, 16.95.

### 5.21. Example 21

### Production of 2-(2-Ethoxy-phenyl)-5-methyl-2-propyl-3H-imidazol-5,1-f][1,2,4]triazin-4-one

A solution of 3-(2-Ethoxy-benzoylamino)-5-methyl-2-propyl-3H-imidazole-4-carboxamide (70 mg, 0.213 mmol) and potassium tert-butoxide (239 mg, 2.13 mmol) in anhydrous tert-butanol (5 mL) was stirred at 160° C. in a sealed tube for 30 h. The cooled reaction mixture was neutralized with 1N HCl before ethyl acetate was added. The organic layer was extracted with water, brine and dried over MgSO₄. After the solvent was removed in vacuo the crude product was purified by flash chromatography on silica gel eluting with 0% increasing to 5% methanol in dichloromethane. The product was obtained as a white solid (48 mg, 72%). ¹H NMR (300 MHz, CDCl₃): δ 9.96 (s_{br}, 1H), 8.18 (dd, ³J=8.1 Hz, ⁴J=1.5 Hz, 1H), 7.51 (t, J=7.6 Hz, 1H), 7.14 (t, J=7.6 Hz, 1H), 7.06 (d, J=8.1 Hz, 1H), 4.27 (q, J=7.1 Hz, 2H), 3.02 (t, J=7.6 Hz, 2H), 2.66 (s, 3H), 1.89 (sextet, J=7.6 Hz, 2H), 1.58 (t, J=7.1 Hz, 3H), 1.05 (t, J=7.6 Hz, 3H). ¹³C NMR (75 MHz, CDCl₃): δ 157.4, 155.3, 146.4, 146.3, 140.2, 133.5, 130.5, 122.1, 117.9, 114.3, 113.5, 65.7, 28.4, 21.4, 15.1, 14.9, 14.4. MS, m/z (%) 313.1 (100) [M⁺+1] Anal. Calcd for C₁₇H₂₀N₄O₂ (312.38): C 65.37, H 6.45, N 17.94. Found: C, 64.99; H, 6.67; N, 17.76.

### 5.22. Example 22

### Production of 7-Bromo-5-methyl-3H-imidazo[5,1-f][1,2,4]triazin-4-one

To a solution of 5-Methyl-3H-imidazo[5,1-f][1,2,4]triazin-4-one (250 mg, 1.66 mmol) in DMF (20 mL) bromine (530 mg, 3.32 mmol) was added at 0° C. The reaction was monitored by TLC and after completion saturated Na₂CO₃-solution (1 mL) was added. The reaction mixture was dried down in vacuo and purified by flash chromatography on silica gel eluting with 2% increasing to 7% methanol in dichloromethane. The product was obtained as a pale yellow solid (203 mg, 53%). ¹H NMR (400 MHz, [D₆]-DMSO): δ 11.82 (s_{br}, 1H), 7.88 (s, 1H), 2.43 (s, 3H). ¹³C NMR (100 MHz, [D₆]-DMSO): δ 154.6, 141.4, 139.9, 118.8, 116.1, 14.7. MS, m/z (%) 230.8 (100) [M⁺+1] Anal. Calcd for C₆H₅BrN₄O (229.04): C 31.46, H 2.20, N 24.46. Found: C, 31.70; H, 2.13; N, 24.27.

### 5.23. Example 23

### Production of 5-Methyl-7-phenyl-3H-imidazo[5,1-f][1,2,4]triazin-4-one

A microwave vial was charged with 7-bromo-5-methyl-3H-imidazo[5,1-f][1,2,4]triazin-4-one (50 mg, 0.218 mmol), phenylboronic acid (26 mg, 0.218 mmol), Pd(dppf)₂Cl₂ (8 mg, 0.010 mmol), 1N Na₂CO₃ (0.436 mL, 0.436 mmol), and acetonitrile (1 mL). After sealing, the vial was degassed and flushed with nitrogen. The suspension was heated to 150° C. for 10 min. The conversion was followed by LC-MS and another equivalent of boronic acid and catalyst was added before the same microwave conditions were applied. Ethayl acetate (10 mL) was added to the clear reaction mixture followed by extraction with water (10 mL) and brine (10 mL). The organic layer was dried over MgSO₄ and the solvent was removed in vacuo. Purification by flash chromatography on silica gel eluting with 75% ethyl acetate in hexane gave the product as a beige-white solid (29 mg, 59%). R_{f} 0.23 (75% EtOAc in hexane), ¹H NMR (300 MHz, [D₆]-DMSO): δ 11.79 (s_{br}, 1H), 8.26 (d, J=7.5 Hz, 2H), 7.92 (s, 1H), 7.48 (m, 3H), 2.54 (s, 3H). ¹³C NMR (75 MHz, [D₆]-DMSO): δ 155.2, 140.7, 139.3, 129.6, 128.9, 128.8, 128.2, 116.9, 14.7. MS, m/z (%) 227.0 (100) [M⁺+1] Anal. Calcd for C₁₂H₁₀N₄O (226.24): C, 63.71; H, 4.46; N, 24.77. Found: C 63.50, H 4.26, N 25.00.

All cited publications, patents, and patent applications are herein incorporated by reference in their entireties.

The present invention is not to be limited in scope by the specific embodiments described herein, which are simply illustrations of individual aspects of the invention. Various modifications of the invention, in addition to those shown and described herein, will become apparent to those skilled in the art from the foregoing description, and are encompassed by the appended claims.

What is claimed is:

1. A method of preparing an imidazotriazinone, which comprises contacting a compound of Formula II 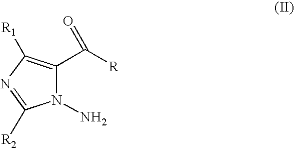
wherein R is alkoxy; R₁ is hydrogen, halogen, alkoxy, nitro, nitrile, optionally substituted alkyl, alkenyl, alkynyl, aryl, aralkyl, or heterocycle; and R₂ is hydrogen, halogen, alkoxy, nitro, nitrile, optionally substituted alkyl, alkenyl, alkynyl, aryl, aralkyl, or heterocycle;
with a compound of formula IV or V 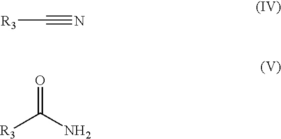
wherein R₃ is hydrogen, optionally substituted lower alkyl, or —N(R₄)(R₅), wherein R₄ and R₅ are individually hydrogen or optionally substituted lower alkyl;
under conditions sufficient for the cyclization of the compound of Formula II.

2. The method of claim 1, wherein R is —OCH₃ or —OCH₂CH₃.

3. The method of claim 1, wherein R₁ is hydrogen, nitrile, or alkyl.

4. The method of claim 1, wherein R₂ is hydrogen or alkyl.

5. The method of claim 1, wherein R₁ is methyl and R₂ is propyl.

6. The method of claim 1, wherein R₃ is hydrogen, —CH₃ or —NH₂.

7. The method of claim 1, further comprising preparing the compound of Formula II by contacting a compound of Formula I 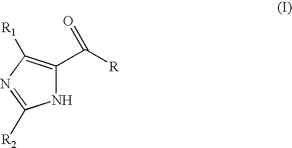
with conditions sufficient to N-aminate the nitrogen atom adjacent to the carbonyl and R₂ moieties.

8. The method of claim 7, wherein the compound of Formula I is N-aminated by contacting it with a base, followed by hydroxylamine-O-sulfonic acid or O-(diphenylphosphinyl)hydroxylamine).

9. A method of preparing an imidazotriazinone, which comprises contacting a compound of Formula II 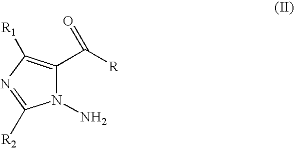
wherein R is amino; R₁ is hydrogen, halogen, alkoxy, nitro, nitrile, optionally substituted alkyl, alkenyl, alkynyl, aryl, aralkyl, or heterocycle; and R₂ is hydrogen, halogen, alkoxy, nitro, nitrile, optionally substituted alkyl, alkenyl, alkynyl, aryl, aralkyl, or heterocycle;
with a compound of formula VI 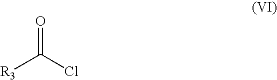
wherein R₃ is hydrogen, optionally substituted alkyl, alkoxy, alkenyl, alkynyl, aryl, aralkyl or heteroaryl;
under conditions sufficient for the cyclization of the compound of Formula II.

10. The method of claim 9, wherein R is —NH₂.

11. The method of claim 9, wherein R₁ is alkyl.

12. The method of claim 9, wherein R₂ is alkyl.

13. The method of claim 9, wherein R₁ is methyl and R₂ is propyl.

14. The method of claim 9, wherein R₃ is optionally substituted aryl.

15. The method of claim 14, wherein R₃ is substituted phenyl.

16. The method of claim 15, wherein R₃ is 2-ethoxy-phenyl.

17. The method of claim 9, further comprising preparing the compound of Formula II by contacting a compound of Formula I 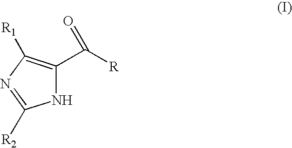
with conditions sufficient to N-aminate the nitrogen atom adjacent to the carbonyl and R₂ moieties.

18. The method of claim 17, wherein the compound of Formula I is N-aminated by contacting it with a base, followed by hydroxylamine-O-sulfonic acid or O-(diphenylphosphinyl)hydroxylamine.

19. A method of preparing vardenafil, which comprises:
contacting 3-(2-ethoxy-benzoylamino)-5-methyl-2-propyl-3H-imidazole-4-carboxamide with conditions sufficient for the formation of 2-(2-ethoxy-phenyl)-5-methyl-7-propyl-3H-imidazo[5,1-f][1,2,4]triazin-4-one;
chlorosulphonation of the 2-(2-ethoxy-phenyl)-5-methyl-7-propyl-3H-imidazo[5,1-f][1,2,4]triazin-4-one to obtain 4-ethoxy-3-(5-methyl-4-oxo-7-propyl-3,4-dihydro-imidazo[5,1-f][1,2,4]triazin-2-yl)-benzenesulfonic acid; and
contacting the 4-ethoxy-3-(5-methyl-4-oxo-7-propyl-3,4-dihydro-imidazo[5,1-f][1,2,4]triazin-2-yl)-benzenesulfonic acid with conditions suitable for the formation of vardenafil.

20. The method of claim 19, which further comprises contacting 3-amino-5-methyl-2-propyl-3H-imidazole-4-carboxamide with 2-ethoxy-benzoyl chloride under conditions sufficient for the formation of the 3-(2-ethoxy-benzoylamino)-5-methyl-2-propyl-3H-imidazole-4-carboxamide.
